(19) United States
(12) Patent Application Publication
(10) Pub. No.: US 2023/0043598 A1
(43) Pub. Date: Feb. 9, 2023

(54) CAPSULE COMPRISING A FAECAL COMPOSITION

(71) Applicant: Hengameh Chloe LAURIDSEN, København SV (DK)

(72) Inventor: Hengameh Chloe LAURIDSEN, København SV (DK)

(21) Appl. No.: 17/788,129

(22) PCT Filed: Dec. 21, 2020
(86) PCT No.: PCT/EP2020/087495

§ 371 Date: Jun. 22, 2022

(30) Foreign Application Priority Data
Dec. 23, 2019 (DK) PA 2019 01545
Nov. 6, 2020 (DK) PA 2020 01259

(51) Int. Cl. A61K 35/74; A61K 47/10; A61K 9/00; A61K 9/19; A61K 9/48

(52) U.S. Cl. CPC A61K 35/74; A61K 47/10; A61K 9/0053; A61K 9/19; A61K 9/4891

(57) ABSTRACT

The present invention relates to a capsule comprising a faecal composition for oral administration wherein the faecal composition comprises a stool fraction and a diluent, wherein the stool fraction comprises one or more non-pathogenic microorganisms, and wherein the diluent comprises a cryoprotectant resulting in a final concentration of cryoprotectant in the faecal composition below 8% (v/v) cryoprotectant.

### TECHNICAL FIELD OF THE INVENTION

The present invention relates to a faecal composition for gut microbiota transplantation. In particular the present invention relates to a faecal composition for gut microbiota transplantation wherein the side effects has been reduced and wherein the daily use is easier and more comfortable.

### BACKGROUND OF THE INVENTION

The human body contains trillions of bacteria cells, fungi, protozoa and viruses, known as the microbiome. The microbiome has been linked to many aspects of human health, from gastrointestinal related diseases to obesity.

Focus on gut microbiota transplantation (GMT), also called faecal microbiota transplantation (FMT), for the prevention and treatment of intestinal disorders has been increasing in the human medicine. increased clinical applications of GMT have provided convincing proofs that modification of the intestinal microbiota is an effective therapy for intestinal dysbiosis-related diseases

Gut microbiota transplantation refers to the process of implanting a faecal suspension from a healthy donor with the right gut microbiota composition into the recipient's intestinal tract to restore the community and function of intestinal microbiota and/or intestinal functionality.

GMT has shown—in randomized, controlled clinical trials—to resolve 80-90% of infections caused by recurrent C. difficile that does not respond to antibiotics.

Though the mechanism of gut microbiota transplantation yet has to be precisely determined. it is believed that GMT, in the treatment of C. difficile infection, works by repopulating the patient's microbiome with diverse microorganisms that competitively exclude C. difficile.

In a healthy gut community, C. difficile is outcompeted or outgrown by many different bacterial species. However, receiving antibiotic treatment disrupts this ecosystem by killing those protective bacteria. C. difficile forms spores that are resistant to antibiotics. No longer outcompeted, antibiotic resistant C. difficile outgrowing in the gut and produces toxins that leave patients suffering from severe diarrhoea, abdominal pain, and, often, fever. With an infusion of intestinal microbiota from a healthy donor, the C. difficile might be competed.

Gut microbiome plays an important role in human and animal health by helping digestion and benefiting the immune system and many other aspects of health. An imbalance of gut microbiota/dysbiosis might be restored by gut microbiota transplantation from a healthy gut, which may contribute to the treatment of various diseases, such as ulcerative colitis and other gastrointestinal conditions.

Jun Hu et al. describes the preparation of faecal compositions where fresh feces are obtained from a healthy donor and diluted with sterile saline and homogenized in a standard blender. The slurry is then filtered three times through gauze, strainer, or 0.25 mm stainless steel sieves to eliminate the undigested and small particulate matter in the faecal suspension. The faecal suspension may be centrifuged at 6,000×g for 15 min. The precipitate, without the supernatant, is re-suspended in fresh sterile saline. The resulting suspension should be transferred to the recipients immediately if a fresh faecal composition is to be used to ensuring faecal microbial viability.

Furthermore, Jun Hu et al. describes preparation of faecal compositions, used for GMT, that may preferably be provided as a frozen faecal suspension which ensures durability, uniformity and viability of the faecal composition and the faecal suspension may be saved for later use. Comparative studies have demonstrated that frozen faecal material does not only simplify the practical steps of clinical human GMT, but also has the similar efficacy to fresh faecal material.

To improve the faecal microbial survival rates during the cryopreservation, fresh stool samples should be diluted with sterile saline homogenized and filtered using the protocol used in the preparation of the fresh faecal material above. Subsequently, the resulting suspension should be added to glycerol (a cryoprotectant) to get a final concentration of 10%. Finally, the faecal suspensions are labeled accurately and then stored, as soon as possible, at −80° C. to ensure the faecal microbial survival. When there is the need to perform a GMT, the frozen faecal suspension should be thawed at and mixed with saline solution to obtain the required concentration and the infusion of faecal suspension should be implemented as soon as possible at room temperature.

The disadvantage of the prior art, exemplified by Jun Hu et al. as described above, is that high amount of glycerol (cryoprotectant) is added to the faecal composition and that other beneficial products in the product, such as fungi, protozoa, viruses, vitamins and minerals produced by the healthy gut microbiota and the nutrition suitable for survival of the healthy gut microbiota in the diseased gut, are discharged together with the supernatant.

Despite the positive effect of cryoprotectant, such as glycerol, on survival rate of cells during cryopreservation, cryoprotectant, such as glycerol, has several disadvantages side effects like being harmful to the mucous membranes; or causing headaches, dizziness, bloating, nausea, vomiting, thirst, or diarrhoea, in particular the negative effects on the mucous membranes by increasing tissue osmolarity, has been found to occur frequently and is undesirable.

Additionally, a further disadvantage of the prior art is the traditional route of transplantation from the donor to the recipient, which is by enema transplantation; probe transplantation or a colonoscopy which are all administered by an injection into the lower bowel via the rectum, e.g. by using a rectal bulb syringe. Even this way of faecal microbiome transplantation has some advantages, the disadvantages is that administration may involve some difficulties, be uncomfortable, intestinal damages/perforations (in particular in the rectum and/or in the sigmoid colon) and/or may require medical assistance.

Hence, an improved faecal composition would be advantageous, and in particular an efficient, a durable, uniform and viable faecal composition for GMT with no or reduced number of side effects, an easier administration, a more comfortable administration and the option of self-administration would be advantageous.

### SUMMARY OF THE INVENTION

Thus, an object of the present invention relates to a faecal composition for gut microbiota transplantation.

In particular, it is an object of the present invention to provide a faecal composition for GMT, that solves the above mentioned problems of the prior art with being harmful to the mucous membranes; or causing headaches, dizziness, bloating, nausea, vomiting, thirst, and/or diarrhoea and at the same time provides an easier administration, a more comfortable administration and the option of self-administration.

Thus, one aspect of the invention relates to a capsule comprising a faecal composition for oral administration, wherein the faecal composition comprises a stool fraction and a diluent, wherein the stool fraction comprises one or more non-pathogenic microorganisms and the diluent comprises a cryoprotectant resulting in a final concentration of cryoprotectant in the faecal composition below 8% (v/v) cryoprotectant.

Another aspect of the present invention relates to a method for providing a capsule comprising a faecal composition, the method comprises the steps of:
(i) obtaining at least one sample of stool from a healthy donor;
(ii) placing said sample obtained in step (i) in a sterile collection device;
(iii) mixing (preferably gently) the sample obtained in step (ii) with at least one diluent comprises a cryoprotectant resulting in a final concentration of cryoprotectant in the faecal composition below 8% (v/v) cryoprotectant;
(iv) filtering the mixture obtained in step (iii), providing the faecal composition comprising a stool fraction and a diluent, wherein the diluent comprises a cryoprotectant resulting in a final concentration of cryoprotectant in the faecal composition below 8% (v/v) cryoprotectant;
(v) filling the faecal composition into a first coating, providing a first coated faecal composition; and
(vi) enclosing the first coated faecal composition with a second coating providing the capsule comprising a faecal composition.

Yet another aspect of the present invention relates to a capsule according to the present invention for use as a medicament.

Still another aspect of the present invention relates to a capsule according to the present invention for use in the treatment and/or prophylaxis of intestinal imbalance, such as chronic Inflammatory Bowel Disease; irritable bowel syndrome; dysbiosis; Clostridium difficile infections; diarrhea; idiopathic constipation; celiac disease; chronic urinary tract infection, type II diabetes; food allergies; cancer; refractory GvHD; obesity and morbid obesity; autism, sclerosis; chronic vaginal infection (including cystitis, mycoses); bone and joint infections; Parkinson's disease; Alzheimer's disease; schizophrenia and bipolar disorders; and gut dysbiosis associated with anti-cancer chemotherapy or immunotherapy.

### DETAILED DESCRIPTION OF THE INVENTION

The inventor of the present invention surprising found that a faecal composition may be provided which has a significant reduction in the side effects and without compromising the efficiency, durability, uniformity and viability of the faecal composition and at the same time provides an easier administration, a more comfortable administration and the option of self-administration.

A preferred embodiment of the present invention relates to a capsule comprising a faecal composition for oral administration wherein the faecal composition comprises a stool fraction and a diluent, wherein the stool fraction comprises one or more non-pathogenic microorganisms and the diluent comprises a cryoprotectant resulting in a final concentration of cryoprotectant in the faecal composition below 8% (v/v) cryoprotectant.

The faecal composition according to the present invention relates to a composition obtained from a human or animal donor, preferably, from a human donor.

In the present context the term “donor” relates to a healthy human or animal—with the suitable gut microbiota delivering a faecal material. In the context of the present invention the term “recipient” relates to a disease human or animal receiving the healthy gut microbiota composition, Vitamins and mineral produced by healthy gut microbiota, antimicrobial peptides, fungi, protozoa, viruses, nutrition necessary for the healthy gut bacteria to survive in the disease prepared from faecal material delivered.

In an embodiment of the present invention the faecal material may be a healthy human or animal faeces or stool, preferably healthy human faeces or stool, with suitable gut microbiota. In the present context the terms “stool” and “faeces” may be used interchangeably.

The stool fraction may comprises one or more non-pathogenic microorganisms. In an embodiment of the present invention the one or more non-pathogenic microorganisms relates to one or more healthy gut bacteria, fungi, protozoa, viruses, present in the faecal material. In an embodiment of the present invention the faecal composition comprises vitamins and mineral produced by healthy gut microbiota, antimicrobial peptides, and nutrition necessary for the healthy gut bacteria to survive in a gut.

In the context of the present invention the term “non-pathogenic microorganism” relates to a microorganism that do not cause disease, harm or death to another organism, such as a human or an animal.

In an embodiment of the present invention at least 50% of the non-pathogenic microorganisms in the faecal composition may be viable non-pathogenic microorganisms relative to the total number of non-pathogenic microorganisms in the faecal composition, such as at least 75%, e.g. at least 90%, such as at least 95%.

In a further embodiment of the present invention the faecal composition may comprise in the range of 10⁴-10¹² viable non-pathogenic microorganisms, such as in the range of 10⁴-10¹², e.g. in the range of 10⁸-10¹⁰, such as in the range of 10⁸-10⁹. Preferably, the viable non-pathogenic microorganisms may be viable non-pathogenic facultative anaerobe, anaerobe and aerobe microorganisms

The faecal composition according to the present invention may be subjected to removal of undigested food. Furthermore, the faecal composition according to the present invention may preferably comprise vitamins and mineral produced by healthy gut microbiota, antimicrobial peptides, and nutrition necessary for the healthy gut bacteria to survive in a gut.

In an embodiment of the present invention a 50-gram stool fraction results in 12-28 gram faecal composition after filtration from undigested food, depending on the diet during the donation time.

In an embodiment according to the present invention the faecal composition does not comprises antibiotics.

In a further embodiment of the present invention the faecal composition according to the present invention comprises no worms; no pathogenic parasites; and/or no pathogenic microorganisms.

Preferably, the faecal composition may not comprise one or more of Clostridium difficile; Clostridium Perfringens, Vibrio, Pleisiomonas, Aeromonas, enteric pathogens, including Salmonella, Shigella; Campylobacter; Escherichia coli 0157 H7, Intimin-producing Escherichia coli, STEC, EPEC, ETEC, EIEC); Carbapenemase-producing enterobacteriae (CPE); Yersinia; vancomycin-resistant enterococci (VRE); methicillin-resistant Staphylococcus aureus (MRSA); Helicobacter; Gram/negative multidrug-resistant bacteria; Enterobacteriaceae, such as Spectrum beta-lactamase (ESBL) producing

Enterobacteriaceae, or carbapenem-resistant Enterobacteriaceae (CRE); Norovirus; Antigens and/or acid-fast staining for Giardia lamblia and Criptosporidium parvum; Protozoa (including Blastocystis hominis) and helminths; and/or faecal occult blood; Entamoeba histolytica, Sapovirus, Rotavirus, Astrovirus, Adenovirus, Coronavirus, worms, worm eggs and cysts, Bacillus cereus.

Preferably, the faecal composition may not comprises above mentioned Clostridium spp.; enteric pathogens, including Salmonella, Shigella; Campylobacter; above mentioned pathogenic Escherichia coli spp.; Yersinia; vancomycin-resistant enterococci (VRE); methicillin-resistant Staphylococcus aureus (MRSA); Gram/negative multidrug-resistant bacteria; Enterobacteriaceae, such as Spectrum beta-lactamase (ESBL) producing Enterobacteriaceae, or carbapenem-resistant Enterobacteriaceae (CRE); Norovirus; Antigens and/or acid-fast staining for Giardia lamblia and Criptosporidium parvum; Protozoa (including Blastocystis hominis) and helminths; and faecal occult blood, Entamoeba histolytica, Sapovirus, Rotavirus, Astrovirus, Adenovirus, Coronavirus, worms, worm eggs and cysts, Bacillus cereus.

In an embodiment of the present invention the faecal composition may be a fresh faecal composition.

In a preferred embodiment of the present invention the faecal may be a frozen faecal composition.

In an embodiment of the present invention the faecal composition may comprise a diluent which is used to make the stool fraction more viscus. In an embodiment of the present invention the diluent may comprise an aqueous solution and/or a cryoprotectant.

The aqueous solution may comprise a salt, a mineral, and acid or an alkaline. Preferably, the aqueous solution comprises a salt, such as sodium chloride, in particular a saline solution. The saline solution comprises sodium chloride, preferably, the concentration of sodium chloride in the saline solution may be 2-30 g/I; such as 4-20 g/I; e.g. 7-19 g/I; such as about 9 g/I sodium chloride (0.9%).

The aqueous solution may preferably be a sterile aqueous solution.

In an embodiment of the present invention the faecal composition may be provided in dosage forms comprising 50 gram stool product and in the range of 5-50 ml saline comprising at most 10% (w/w) glycerol, such as at most 8% (w/w) glycerol, e.g. at most 5% (w/w) glycerol; such as at most 4% (w/w) glycerol, e.g. at most 3% (w/w) glycerol; such as at most 2% (w/w) glycerol, e.g. at most 1% (w/w) glycerol, relative to the saline solution; such as in the range of 10-40 ml saline, e.g. in the range of 12-35 ml saline, such as in the range of 15-30 ml saline, e.g. about 20 ml saline.

Preferably, the faecal composition according to the present invention may be provided in dosage forms consisting essentially of:
50 gram stool product; and
in the range of 5-50 ml saline comprising at most 10% (w/w) glycerol, such as at most 8% (w/w) glycerol, e.g. at most 5% (w/w) glycerol; such as at most 4% (w/w) glycerol, e.g. at most 3% (w/w) glycerol; such as at most 2% (w/w) glycerol, e.g. at most 1% (w/w) glycerol, relative to the saline solution; such as in the range of 10-40 ml saline, e.g. in the range of 12-35 ml saline, such as in the range of 15-30 ml saline, e.g. about 20 ml saline.

In the context of the present invention the term “stool product” relates to stool present in the faecal composition where undigested and small particulate material have been reduced or removed.

The amount of diluent, such as a saline containing diluent (or diluent comprising cryoprotectant, preferably glycerol) may depend on the stool type. A stool having the Bristol stool chart type 2 may be mixed with about 30 ml saline comprising at most 10% (w/w) glycerol, such as at most 8% (w/w) glycerol, e.g. at most 5% (w/w) glycerol; such as at most 4% (w/w) glycerol, e.g. at most 3% (w/w) glycerol; such as at most 2% (w/w) glycerol, e.g. at most 1% (w/w) glycerol; whereas a stool having the Bristol stool chart type 4 may be mixed with about 15 ml saline comprising at most 10% (w/w) glycerol, such as at most 8% (w/w) glycerol, e.g. at most 5% (w/w) glycerol; such as at most 4% (w/w) glycerol, e.g. at most 3% (w/w) glycerol; such as at most 2% (w/w) glycerol, e.g. at most 1% (w/w) glycerol.

In an embodiment of the present invention the diluent may be added to the stool in several sequences, such as addition in 2 sequences, e.g. addition in 3 sequences, such as addition in 4 sequences.

In a further embodiment of the present invention addition of diluent to the stool fraction may be done by an initial addition of a sterile saline solution to increase viscosity followed by addition of a cryoprotectant before freezing. The initial addition of saline solution may involve several additions of saline solutions.

In yet an embodiment of the present invention the sterile cryoprotectant may be added to the stool fraction in combination with an sterile aqueous solution, such as a saline solution.

In a preferred embodiment of the present invention the diluent comprises a cryoprotectant resulting in a final concentration of cryoprotectant in the faecal composition below 8% (v/v) cryoprotectant; such as 7% (v/v) cryoprotectant or below; e.g. 6% (v/v) cryoprotectant or below; such as 5% (v/v) cryoprotectant or below; e.g. 4% (v/v) cryoprotectant or below; such as 3% (v/v) cryoprotectant or below; e.g. 2% (v/v) cryoprotectant or below; such as 1% (v/v) cryoprotectant or below; e.g. 0.5% (v/v) cryoprotectant or below; such as 0.1% (v/v) cryoprotectant or below; e.g. 0.05% (v/v) cryoprotectant or below; such as 0.001% (v/v) cryoprotectant or below; e.g. 0.0001% (v/v) cryoprotectant or below; such as in the range of 0-8% (v/v) cryoprotectant; e.g. in the range of 0.0001-7% (v/v) cryoprotectant; such as in the range of 0.001-6% (v/v) cryoprotectant; e.g. in the range of 0.01-5% (v/v) cryoprotectant; such as in the range of 0.1-4% (v/v) cryoprotectant; e.g. in the range of 1-3% (v/v) cryoprotectant; such as in the range of 1.5-2.5% (v/v) cryoprotectant; e.g. in the range of 0.1-2% (v/v) cryoprotectant.

In an embodiment of the present invention the content of the diluent may constitute less than 5 times the content of the stool fraction on a (w/w) basis; such as less than 4 times the content of the stool fraction on a (w/w) basis; e.g. less than 3 times content of the content of the stool fraction on a (w/w) basis; such as less than 2.5 times the content of the stool fraction on a (w/w) basis; e.g. less than 2 times content of the content of the stool fraction on a (w/w) basis; such as less than 1.8 times the content of the stool fraction on a (w/w) basis; e.g. less than 1.6 times content of the content of the stool fraction on a (w/w) basis; such as less than 1.4 times the content of the stool fraction on a (w/w) basis; e.g. less than 1.2 times content of the content of the stool fraction on a (w/w) basis.

In a further embodiment of the present invention the cryoprotectant may be selected from the group consisting of glycerol; mannitol; sorbitol; propylene glycol; ethylene glycol; trehalose and its analogues; saccharose; galactose-lactose and mixtures hereof.

Preferably, the cryoprotectant may be glycerol.

In an embodiment of the present invention the faecal composition present in the capsule may be a freeze dried or frozen faecal composition, preferably, a frozen faecal composition.

In yet an embodiment of the present invention the faecal composition present in the capsule is a liquid faecal composition. Preferably, the capsule comprises within 0.1-1 gram liquid faecal composition per capsule.

In a further embodiment of the present invention the capsule (comprising fresh faecal composition or liquid faecal composition) may be frozen. Preferably, the capsule may be frozen after the faecal composition has been added coated with at least a first coating, preferably coated with a first coating and a second coating.

The stool fraction and/or the faecal composition may comprise a particulate material such as beneficial bacteria such as: Faecalbacterium, Prevotella bacteria, Lactobacilli species, Weissella spp., Bifidobacterium spp., Streptococcus thermophilus. The stool fraction also comprises antimicrobial peptides, healthy gut microbiota composition, short chain fatty acid, which serves as nutrition for host cell functionality (only produced by healthy gut microbiota), vitamins and minerals produced by healthy gut microbiota, fungi, protozoa, viruses, nutrition necessary for the healthy gut bacteria to survive in the disease.

The particulate material may comprise a particle size of the stool fraction and/or the faecal composition is below 0.4 mm; such as 0.3 mm or below; e.g. 0.25 mm or below; such as 0.20 mm or below.

In an embodiment of the present invention the capsule may comprises a faecal composition, wherein the faecal composition may be a liquid faecal composition.

In an embodiment of the present invention the stool fraction may be provided from a stool obtained from a mammal (donor). The mammal may be a human; a dog; a cat; a horse; cattle; sheep; goat; a chicken; a duck; and a pig.

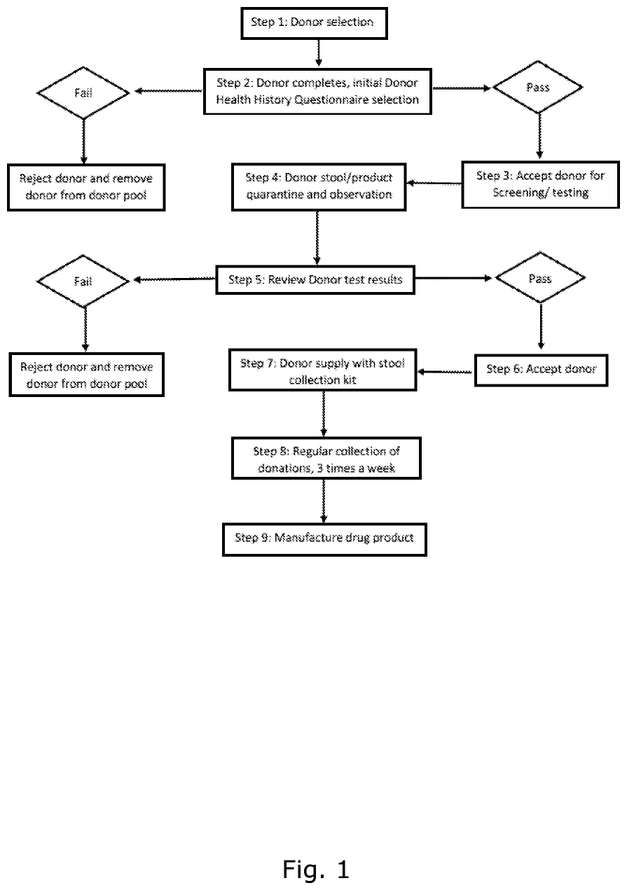

The selection of a suitable and healthy stool-donor may follow a very strict scheme for collecting and inspecting donor faecal sample to ensure quality of the stool. The process of donor selection may be described below and may be demonstrated in the FIG. 1.

Initially the donor should pass the preliminary donor health questionnaire regarding various health questions.

The donor stool may be obtained and tested. If approved (e.g. not comprising pathogenic microorganisms, antibiotics, presence of medicine, etc.) the donor may be accepted.

In an embodiment the donor stool may be placed in quarantine for 3 months at 5-8 degrees to investigate if the donor intestinal bacteria produce any toxins in the stool. If the donor is approved the donor may be supplied with a stool collection kit, e.g. comprising a zip-bag for collecting the stool until processed.

Preferably the donor may deliver stool 3 times a week. Once the donor is accepted, they will be supplied with the collection kit and they must regularly donate up to 3 times a week.

The quality of the stool may be checked frequently, such as 4 times a year. E.g. once a year, such as each second month, e.g. each month, such as each 14 days, e.g. each week, e.g. every time a stool donation is made.

In an embodiment of the present invention, the capsule comprises between 0.1-5 gram faecal composition per capsule, such as 0.5-2.5 gram faecal composition per capsule, e.g. 0.75-1.5 gram faecal composition per capsule, such as about 1 gram faecal composition per capsule.

In an embodiment of the present invention the capsule may comprise a first coating and a second coating. The first coating may be surrounding the faecal composition and the second coating may be surrounding the first coating.

The first coating may preferably be a pH responsive polymer composition. The first coating may provide a delayed release of the faecal composition. The term “delayed release” may be a release after at least 10 minutes, such as at least 15 minutes, e.g. at least 20 minutes, such as at least 25 minutes, e.g. at least 30 minutes, such as within a period of 10-50 minutes, e.g. for a period of 20-40 minutes, such as for a period of about 30 minutes.

In an embodiment of the present invention the pH responsive polymer composition may comprise HPMC (hydroxypropyl methyl cellulose empty hard capsules). The first coating may preferably have a fill size 0.

The first coating may preferably be surrounded by a second coating. The second coating may dissolve within a period of time in the range of 5-25 minutes, such as in the range of 10-20 minutes, e.g. in about 15 minutes.

The second coating may preferably dissolve in the stomach of the recipient and the first coating may dissolve or provide a delayed release of the faecal composition to the intestine.

In an embodiment of the present invention the second coating may comprise HPMC

(Vegetable Capsules, hydroxypropyl methyl cellulose empty hard capsules). The second coating may preferably have a fill size 00.

A preferred embodiment of the present invention relates to a method for providing a capsule comprising a faecal composition, the method comprises the steps of:
(i) obtaining at least one sample of stool from a healthy donor;
(ii) placing said sample obtained in step (i) in a sterile collection device;
(iii) mixing the sample obtained in step (ii) with at least one diluent comprises a cryoprotectant resulting in a final concentration of cryoprotectant in the faecal composition below 8% (v/v) cryoprotectant (in particular 5% cryoprotectant, e.g. glycerol, in combination with 0.9% saline);
(iv) filtering the mixture obtained in step (iii), providing the faecal composition comprising a stool fraction and a diluent, wherein the diluent comprises a cryoprotectant resulting in a final concentration of cryoprotectant in the faecal composition below 8% (v/v) cryoprotectant;
(v) enclosing the faecal composition into a first coating, providing a first coated faecal composition; and
(vi) enclosing the first coated faecal composition with a second coating providing the capsule comprising a faecal composition.

The healthy donor may be analyzed and determined to be an acceptable healthy donor as described herein.

The filtering performed in step (iv) may be performed using at least one filter having pores of diameter less than or equal to 0.4 mm, such as less than or equal to 0.3 mm, e.g. less than or equal to 0.25 mm, such as less than or equal to 0.20 mm.

Preferably, the filtering performed in step (iv) may be performed in a two-step filtering procedure. In an embodiment of the present invention the two-step filtering procedure involves a first filtering process using a filter with a pore size in the range of 0.5-1 mm and/or a second filtering process using a filter with a pore size in the range of 0.01-0.25 mm.

The supernatant obtained from the filtering performed in step (iv) may be included as part of the faecal composition.

In an embodiment of the present invention the faecal composition is not added a water immiscible compound. Preferably, the water immiscible compound is an oil compound.

Polyols, like glycerol, may be a water miscible compound.

In an embodiment of the present invention the faecal composition is not added a carbohydrate, an oligosaccharide or a polysaccharide compound.

In an embodiment of the present invention the faecal composition comprises:
(1) supernatant obtained from the filtering performed in step (iv); antimicrobial peptides, short chain fatty acid, vitamins and/or minerals;
(2) microbiota, fungi, protozoa, viruses, and/or nutrition for bacteria to survive in disease gut;
(3) glycerol;
(4) saline compound; and the faecal composition does not comprise
(5) added oil: and
(6) added carbohydrate, oligosaccharide or polysaccharide compound

In an embodiment of the present invention the enclosing of the faecal composition into a first coating, providing a first coated faecal composition as provided in step (v) may be performed by filling the faecal composition into a first coating, providing a first coated faecal composition.

To improve viability and durability of the faecal composition the faecal composition may be stored for at least 6 hours at a temperature of −80° C. or less, such as for at least 12 hours, e.g. for at least 24 hours, such as for at least 3 days, e.g. for at least 5 days, such as for at least 7 days, e.g. for at least 10 days, such as for at least 15 days, e.g. for at least 25 days, such as for at least 1 month, e.g. for at least 3 months, such as for at least 6 months, e.g. for at least 9 months, such as for at least 12 months or 2 till 3 years.

The faecal composition may be immediately stored at a temperature of −80° C. or less.

In an embodiment of the present invention the stool desired for the purpose of the present invention may be characterised as a type 2-4 on the Bristol Stool Chart; such as a type 2-3; e.g. a type 2-4; such as a type 3-4; e.g. a type 4. According to the present invention, the stool belonging to the type 1, 6 of 7 of the Bristol Stool Chart may be controlled (to a higher extent) for the presence of pathogenic microorganisms, e.g. bacteria, before use.

In order to ensure a high-quality stool sample provided in step (i) a donor suitable for delivering may be tested (preferably from blood samples) for one or more, preferably all, of:
Cytomegalovirus, IgM, IgG
Epstein-Barr virus IgM
Treponema Pallidum IgG, IgM
Hepatitis A
HBV, Hepatitis B virus
HCV, Hepatitis C virus
Hepatitis E virus
Syphilis
HIV-1 and HIV-2
Corona virus
Entamoeba histolytica
Complete blood cell count with differential:
   Hematocrito, Hemoglobin, VCM, HCM, CHCM, Platelets, Leukocyte, Differential Leukocyte count
C-reactive protein and erythrocyte sedimentation rate
Coagulation factor, APTT
IgG, IgA, IgM
Albumin
Creatinine and electrolytes (Calcium, K, Na, CI, AST, ALT)
Aminotransferases, bilirubin, gamma-glutamyltransferase, alkaline phosphatase Glucose, Cholesterol, Triglyceride, HDL, LDL, TSH
Human T-lymphotropic virus types I and II antibodies
Strongyloides stercoralis

In order to further ensuring a high-quality stool sample provided in step (i) the stool from a donor suitable for delivering the stool sample may be tested for one or more, preferably all, of:
Detection of Clostridium difficile; Clostridium Perfringens
Detection of enteric pathogens, including Salmonella, Shigella
Campylobacter, Escherichia coli (0157 H7, Intimin-producing Escherichia coli, STEC, EPEC, ETEC, EIEC), Yersinia, vancomycin-resistant enterococci (VRE), methicillin-resistant Staphylococcus aureus (MRSA, nasal or peri-rectal swabs is also ok if not stool), Gram/negative multidrug-resistant bacteria, Spectrum beta-lactamase (ESBL) producing Enterobacteriaceae, carbapenem-resistant Enterobacteriaceae (CRE); Carbapenemase-producing enterobacteriae (CPE)
Norovirus
Antigens and/or acid-fast staining for Giardia lamblia and Criptosporidium parvum
Protozoa (including Blastocystis hominis) and helminths
Faecal occult blood testing
Detection of Vibrio Cholera and Listeria monocytogens
Antigens and/or acid-fast staining for Isospora and Microsporidia
Calprotectin
Helicobacter pylori faecal antigen
Rotavirus
Pleisiomonas
Aeromonas,
Entamoeba histolytica
Sapovirus,
Astrovirus,
Adenovirus,
Corona virus
worms, worm eggs and cysts,
Bacillus cereus,

Preferably stool from the donor should be tested on a regularly basis, such as each 2^{nd} months, preferably, for a period of at least 6 months after stool donation, e.g. at least 8 months, such as at least 10 months, e.g. at least 12 months, such as at least 18 months.

In order to improve viability of the faecal composition or for increasing storage of the faecal composition according to the present invention, the faecal composition be stored (preferably immediately) at a temperature of −80° C. or less, such as a temperature of −100° C. or less, e.g. a temperature of −120° C. or less, such as a temperature of −150° C. or less.

In an embodiment of the present invention the process of freezing the faecal composition to a temperature of −100° C. or less, e.g. a temperature of −120° C. or less, such as a temperature of −150° C. or less, may be performed in a stepwise procedure comprising a first freezing step to about −80 C followed by a second freezing step to a temperature of −120° C. or less, such as a temperature of −150° C. or less.

The faecal composition may be stored at the final storage temperature for a period of at least 40 minutes, e.g. at least 1 hour, e.g. at least 24 hours, e.g. at least 1 week, e.g. at least 2 weeks, e.g. at least 1 month, e.g. at least 3 months, e.g. at least 5 months, e.g. at least 1 year, e.g. at least 2 years, e.g. at least 3 years, preferably about 1 year.

In an embodiment of the present invention the method, after the sample obtained in (i) has been places in the sterile collection device (step (iii), is performed anaerobically. The sterile collection device may be a zip bag or a container.

In an embodiment of the present invention the mixing of the sample obtained in (ii) with at least one diluent (step (iii)) may be performed manually or mechanically using a grinder or a blender. A grinder may be preferred.

A preferred embodiment of the present invention relates to a capsule according to the present invention for use as a medicament.

A further preferred embodiment of the present relates to a capsule according to the present invention for use in the treatment and/or prophylaxis of intestinal imbalance, such as chronic Inflammatory Bowel Disease; irritable bowel syndrome; dysbiosis; Clostridium difficile infections; diarrhea; idiopathic constipation; celiac disease; chronic urinary tract infection, type II diabetes; food allergies; cancer; refractory GvHD; obesity and morbid obesity; autism, sclerosis; chronic vaginal infection (including cystitis, mycoses); bone and joint infections; Parkinson's disease; Alzheimer's disease; schizophrenia and bipolar disorders; and gut dysbiosis associated with anti-cancer chemotherapy or immunotherapy.

In the context of the present invention the term “intestinal induced disease” relates to an imbalance in the gastrointestinal track, such as in the intestine caused by an inflammation in the gastrointestinal track, such as in the intestine, or in one or more parts of the intestine.

The disease caused by an imbalance in the intestine, e.g. intestinal induced disease, may be selected from the group consisting of chronic Inflammatory Bowel Disease; irritable bowel syndrome; dysbiosis; Clostridium difficile infections; diarrhea; idiopathic constipation; celiac disease; chronic urinary tract infection; type II diabetes; food allergies; cancer; refractory GvHD; obesity and morbid obesity; autism, sclerosis; constipation; chronic urinary tract infection or vaginal infection (including cystitis, mycoses); bone and joint infections; Parkinson's disease; Alzheimer's disease; schizophrenia and bipolar disorders; and gut dysbiosis associated with anti-cancer chemotherapy or immunotherapy.

This imbalance may be caused by an inflammation in the intestine or in one or more parts of the intestine.

In an embodiment of the present invention an inflammation in the small intestine of a mammal, such as a human, may be treated and/or suppressed by administering to the mammal, such as a human, an effective amount of the faecal composition by oral administration. Preferably, the faecal composition administered to the small intestine by oral administration may be provided in the form of capsules.

In another embodiment of the present invention an inflammation in the intestinal appendix of a mammal, such as a human, may be treated and/or suppressed by administering to the mammal, such as a human, an effective amount of the faecal composition by oral administration. Preferably, the faecal composition administered to the intestinal appendix by oral administration may be provided in the form of capsules.

In yet an embodiment of the present invention an inflammation in the cecum of a mammal, such as a human, may be treated and/or suppressed by administering to the mammal, such as a human, an effective amount of the faecal composition by oral administration. Preferably, the faecal composition administered to the cecum by oral administration may be provided in the form of capsules.

In an embodiment of the present invention the capsules may also be administered in combination with one or more immunosuppressive medication.

In another embodiment of the present invention an inflammation in the ascending colon of a mammal, such as a human, may be treated and/or suppressed by administering to the mammal, such as a human, an effective amount of the faecal composition by oral administration. Preferably, the faecal composition administered to the ascending colon by oral administration may be provided in the form of capsules.

In yet an embodiment of the present invention an inflammation in the transverse colon of a mammal, such as a human, may be treated and/or suppressed by administering to the mammal, such as human, an effective amount of the faecal composition by oral administration. Preferably, the faecal composition administered to the transverse colon by oral administration may be provided in the form of capsules.

In yet an embodiment of the present invention an inflammation in the descending colon of a mammal, such as a human, may be treated and/or suppressed by administering an effective amount of the faecal composition by oral administration of the faecal composition. Preferably, the faecal composition for the oral administration may be provided in the form of capsules.

In a further embodiment of the present invention an inflammation in the sigmoid colon of a mammal, such as a human, may be treated and/or suppressed by administering an effective amount of the faecal composition by oral administration of the faecal composition. Preferably, the faecal composition for the oral administration may be provided in the form of capsules.

An inflammation in the small intestine, in the intestinal appendix, in the cecum, in the ascending colon, in the transverse colon; in the descending colon; and/or in the sigmoid colon of a mammal, such as a human, may be treated and/or suppressed by administering to the mammal, such as human, an effective amount of the faecal composition by oral administration. Preferably, the faecal composition administered to the in the small intestine, in the intestinal appendix, in the cecum, in the ascending colon, and/or in the transverse colon by oral administration may be provided in the form of capsules.

It should be noted that embodiments and features described in the context of one of the aspects of the present invention also apply to the other aspects of the invention.

The invention will now be described in further details in the following non-limiting examples.

### EXAMPLES

### Example 1

A fresh faecal composition was prepared following the below procedure:
1. A healthy donor with suitable intestinal microbiota was selected, and tested according to the present invention. A fresh stool sample was obtained from approved donors and subjected to a first microbial analysis,
2. Depending the stool type (Brisol-stool-chart), 10 to 30 ml sterile anaerobic saline containing below 8% glycerol was added to a zip bag/Bagpage XRCk containing 50-gram fresh stool and treated anaerobically.
3. The fresh stool sample (comprising the saline solution) was mixed manually for 5 minutes, but a BagMixer could also have been used
4. The mixture was gradually filtered down to <1.25 millimeter resulting in a fresh faecal composition.

### Example 2

A capsule comprising the faecal composition as provided in Example 1 was prepared immediately following the below procedure:
5. The filtered and fresh faecal composition obtained from step 5 of example 1 is filled into a sterile pipette or 30 ml syringe
6. using the sterile pipette or the syringe in step 7 was used to fill a size 0 fillable Amber-Tinted Empty vegetarian Capsule with 950 mg faecal composition and the capsule was closed.
7. The filled capsule from step 8 was insert it in size 00 fillable vegetarian Capsule and immediately closed.
8. The capsule is stored at −80° C. regularly while processing.
9. The capsules comprising the faecal composition can be stored at −80° C. for at least a year.
10. during treatment using the capsules produced a dosage of 10-15 capsules according to the present invention should be taken orally as frozen with at least 500 ML water, preferably as the first thing in the morning.

### REFERENCES

Jun Hu et al. Front Microbiol. 2018; 9:1328
(https://www.ncbi.nlm.nih.gov/pmc/articles/PMC6018536/)

1. A capsule comprising a faecal composition for oral administration wherein the faecal composition comprises a stool fraction and a diluent, wherein the stool fraction comprises one or more non-pathogenic microorganisms, and the diluent comprises a cryoprotectant resulting in a final concentration of cryoprotectant in the faecal composition below 8% (v/v) cryoprotectant.

2. The capsule according to claim 1, wherein the diluent comprises below 8% (v/v) cryoprotectant; such as 7% (v/v) cryoprotectant or below; e.g. 6% (v/v) cryoprotectant or below; such as 5% (v/v) cryoprotectant or below; e.g. 4% (v/v) cryoprotectant or below; such as 3% (v/v) cryoprotectant or below; e.g. 2% (v/v) cryoprotectant or below; such as 1% (v/v) cryoprotectant or below; e.g. 0.5% (v/v) cryoprotectant or below; such as 0.1% (v/v) cryoprotectant or below; e.g. 0.05% (v/v) cryoprotectant or below; such as 0.001% (v/v) cryoprotectant or below; e.g. 0.0001% (v/v) cryoprotectant or below; such as in the range of 0-8% (v/v) cryoprotectant; e.g. in the range of 0.0001-7% (v/v) cryoprotectant; such as in the range of 0.001-6% (v/v) cryoprotectant; e.g. in the range of 0.01-5% (v/v) cryoprotectant; such as in the range of 0.1-4% (v/v) cryoprotectant; e.g. in the range of 1-3% (v/v) cryoprotectant; such as in the range of 1.5-2.5% (v/v) cryoprotectant; e.g. in the range of 0.1-2% (v/v) cryoprotectant.

3. The capsule according to claim 1, wherein the faecal composition present in the capsule is a liquid faecal composition.

4. The capsule according to claim 1, wherein the cryoprotectant is selected from the group consisting of glycerol; mannitol; sorbitol; propylene glycol; ethylene glycol; trehalose and its analogues; saccharose; galactose-lactose and mixtures hereof. Preferably, the cryoprotectant is glycerol.

5. The capsule according to claim 1, wherein the capsule is frozen.

6. The capsule according to claim 1, wherein the capsule comprises between 0.1-5 gram faecal composition per capsule, such as 0.5-2.5 gram faecal composition per capsule, e.g. 0.75-1.5 gram faecal composition per capsule, such as about 1 gram faecal composition per capsule.

7. The capsule according to claim 1, wherein the capsule comprises a first coating and a second coating, wherein the first coating is surrounding the faecal composition and the second coating is surrounding the first coating.

8. A method for providing a capsule comprising a faecal composition, the method comprises the steps of:
(i) obtaining at least one sample of stool from a healthy donor;
(ii) placing said mentioned sample obtained in step (i) a sterile collection plastic device;
(iii) mixing the sample obtained in step (ii) with at least one sterile diluent comprises a cryoprotectant resulting in a final concentration of cryoprotectant in the faecal composition below 8% (v/v) cryoprotectant;
(iv) filtering the mixture obtained in step (iii), providing the faecal composition comprising a stool fraction and a diluent, wherein the diluent comprises a cryoprotectant resulting in a final concentration of cryoprotectant in the faecal composition below 8% (v/v) cryoprotectant;
(v) filling the faecal composition into a first coating, providing a first coated faecal composition; and
(vi) enclosing the first coated faecal composition with a second coating providing the capsule comprising a faecal composition.

9. A capsule according to claim 1, for use as a medicament.

10. A capsule according to claim 1, for use in the treatment and/or prophylaxis of intestinal imbalance, such as chronic Inflammatory Bowel Disease; irritable bowel syndrome; dysbiosis; Clostridium difficile infections; diarrhea; idiopathic constipation; celiac disease; chronic urinary tract infection; type II diabetes; food allergies; cancer; refractory GvHD; obesity and morbid obesity; autism, sclerosis; chronic vaginal infection (including cystitis, mycoses); bone and joint infections; Parkinson's disease; Alzheimer's disease; schizophrenia and bipolar disorders; and gut dysbiosis associated with anti-cancer chemotherapy or immunotherapy.
